(19) United States
(12) Patent Application Publication
(10) Pub. No.: US 2006/0051369 A1
(43) Pub. Date: Mar. 9, 2006

(54) ADAR1 antiviral pathway

(76) Inventors: Deborah Rayne Taylor, Frederick, MD (US); Stephen M. Feinstone, Washington, DC (US); Montserrat Puig, Bethesda, MD (US); Kathleen Mihalik, Oak Hill, VA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/212,258

(22) Filed: Aug. 26, 2005

Related U.S. Application Data
(60) Provisional application No. 60/605,238, filed on Aug. 27, 2004.
(60) Provisional application No. 60/668,763, filed on Apr. 5, 2005.

(51) Int. Cl. A61K 39/12; C12N 7/00; C12N 15/86

(52) U.S. Cl. 424/199.1; 435/456; 435/235.1

(57) ABSTRACT

Methods for enhancing the production of viral vaccines in cell culture are described, as are the cell cultures. These methods rely on the suppression of the levels of ADAR1 in interferon-deficient cells. The methods comprise (a) infecting a cell culture with a donor virus, wherein said cell culture is an interferon-deficient cell culture that is deficient in ADAR1 activity; (b) culturing said infected cell culture under conditions sufficient to provide efficient virus growth; and (c) harvesting the virus produced.

### RELATED APPLICATIONS

This application claims the benefit of priority of U.S. Provisional application No.: 60/605,238, filed Aug. 27, 2004, and U.S. Provisional application No.: 60/668,763, filed Apr. 5, 2005, both of which are hereby expressly incorporated by reference in their entireties.

### FIELD OF THE INVENTION

The present invention relates to methods for the production of virus for vaccines in cell culture.

### BACKGROUND OF THE INVENTION

HCV infects approximately 170 million individuals worldwide and nearly 3 million in the United States alone. Most cases of HCV become persistent and may result in chronic liver disease, cirrhosis and hepatocellular carcinoma. The current combination antiviral therapy of is pegylated interferon-α (IFN-α) with ribavirin is effective in approximately 50 percent of individuals treated while monotherapy with IFN-α alone is successful in less than 20 percent of patients (McHutchison, J G et al. 1998 N Eng. J Med 339:1485-1492). IFN-α allows cells to become innately primed for defense against eventual virus attack by inducing the transcription of many genes, some of which are activated during virus infection. Only a few genes have been identified and characterized as mediators of the IFN-α-induced antiviral response, including the Mx proteins, major histocompatibility complex proteins, 2,5′ oligoadenylate synthetase (2,5 OAS), and the double-stranded (ds) RNA-activated protein kinase (PKR)(Taylor D R 2000 J Mol Med 78:182-190). PKR is activated during viral infection, which results in the phosphorylation of the alpha subunit of the translation-initiation factor, eIF2, and subsequent translational shut-off. Adenosine deaminase that acts on dsRNA (ADAR1) is also IFN-α induced and catalyzes the deamination of adenosine residues in dsRNA (for review, see Saunders, L R and Barber, G N 2003 FASEB J 17:961-983), resulting in inosine substitution. Inosine residues are not abundantly found in cellular mRNAs, but when present are transcribed and translated as guanosine residues, which may lead to mutations (Bass, B L 1997 Trends Biochem Sci 22:157-162; Wang, Q et al. 2000 Science 290:1765-1768). An RNase that specifically degrades inosine-containing RNA has been described and was proposed to be part of a putative antiviral pathway (Scadden, A D J and Smith, C W J 1997 EMBO J 16:2140-2149; Scadden, A D J and Smith, C W J 2001 EMBO J 20:4243-4252). Although antiviral activity has not been attributed to ADAR1, Hepatitis Delta virus (HDV) utilizes ADAR1 editing to promote its viral life cycle (Polson, A G et al. 1996 Nature 380:454-456).

Typically, dsRNA is found only in cells that are virus infected, and both DNA and RNA viruses may present dsRNA in the cell in the form of replicative intermediates (Maran, A and Mathews, M B 1988 Virology 164:106-113). ADAR1 contains three copies of a conserved dsRNA-binding motif (dsRBM) also found in PKR (Wong, S K and Lazinski, D W 2002 Proc Natl Acad Sci USA 99:15118-15123). Most viruses have developed strategies to evade the effects of IFN. For instance, a single-stranded virus-encoded RNA with partially dsRNA features, adenovirus-associated (VA) RNA_{I}, enhances translation and counteracts the effects of IFN in adenovirus-infected cells by inhibiting PKR (O'Malley, R P et al. 1986 Cell 44:391-400). VA RNA has also been shown to bind and inhibit ADAR1 (Lei, M et al. 1998 Virology 245:188-196).

### SEGUE TO THE INVENTION

This is the first report identifying RNA editing by ADAR1 in the control of viral replication and serves as the basis of a potent new strategy for an effective treatment against HCV based on ADAR1 activation.

### SUMMARY OF THE INVENTION

Methods for enhancing the production of viral vaccines in cell culture are described, as are the cell cultures. These methods rely on the suppression of the levels of ADAR1 in interferon-deficient cells. The methods comprise (a) infecting a cell culture with a donor virus, wherein said cell culture is an interferon-deficient cell culture that is deficient in ADAR1 activity; (b) culturing said infected cell culture under conditions sufficient to provide efficient virus growth; and (c) harvesting the virus produced.

### BRIEF DESCRIPTION OF THE DRAWINGS

FIG. 1. Interferon (IFN)-induced biochemical pathways affecting cell growth and metabolism. Among the many proteins induced by IFNs are double-stranded RNA-activated protein kinase (PKR), P56, adenosine deaminase that acts on double-stranded RNA (ADAR), and 2′-5′-oligoadenylate synthetase (2-5(A) synthetase).

FIG. 2. IFN-α action is replicon specific. (A) Immunoblot of HCV NS3 protein expression in lysates (25 μg) from parental Huh7 cells or replicon-containing cells (Huh.BB7) treated with IFN-α (100 IU/mL) for 24, 48 or 72 hours. (B) Total cellular proteins from an equal number of ³⁵S-methionine metabolically labeled cells after treatment with IFN-α (100 IU/mL) for 24, 48 or 72 hours. (C) HCV RNA from 0.5×10⁶ cells was quantitated by Taqman RT-PCR using HCV standards. Total RNA from the same cells was measured by UV₂₆₀ absorbance.

FIG. 3. Cap-independent translation exhibits IFN-α resistance. (A) Bicistronic luciferase reporters expressing Renilla luciferase (luc) under control of a cap-dependent translation mechanism and firefly luciferase under the EMCV- or HCV-IRES. (B, C) Luc activity expressed as fold-inhibition as a result of IFN-α treatment in cell lysates from Huh7 or Huh.BB7 cells transfected with bicistronic reporter plasmid DNA (2 μg) containing the EMCV IRES (E) or HCV (H) IRES constructs shown in panel A, expressed as relative to the control (no IFN-α) (which was assigned a value of 10). (D and E) IFN-α (100 IU/mL) inhibition of luciferase activity in cell lysates from equal numbers of Huh.BB7 or Huh7 cells transfected with bicistronic reporter plasmid RNA (synthesized in vitro using T7 RNA polymerase) containing the EMCV (E) or HCV (H) IRES. Luc assays shown are representative of 4 or more experiments with transfections performed in duplicate and assayed for luc activity in triplicate. Transfection efficiency was also monitored by RNase protection analysis. Error bars represent the standard deviation of the mean (±SD). (F) Summary of data panels B to E.

FIG. 4. Inhibition of PKR stimulates translation but does not result in complete rescue of the replicon from the effects of IFN-α. (A) Luciferase activity measured in cell lysates from Huh.BB7 cells transfected with HCV IRES bicistronic reporter plasmid (2 μg) and E2 and/or pcDNA3 plasmids (10 μg total DNA/6 cm dish). Cells were untreated [(−)IFN]or treated with 1,500 IU/mL IFN-α [(+)IFN] for 18 hr. The expression levels of transfected E2 are shown below the bars. Immunoblot of E2 from transfected cells. RNA was isolated from transfected cells and luc expression/transfection efficiency was monitored by RNase protection analysis (RPA) as described in Examples. (B) Luciferase activity in cells cotransfected with HCV IRES bicistronic reporter plasmid and E2 or NS5A (6 μg) with pcDNA3 (6 μg) or with both E2 and NS5A (E+N; 6 μg each). Luciferase activity is expressed as the fold-increase. Error bars represent ±SD. (C) Huh.BB7 cells transfected in triplicate with E2, NS5A or pcDNA3 (vector). Cells were untreated (0) or treated with 100 IU/ml IFN-α at 8 hr post-transfection (72 hr), 32 hr post-transfection (48 hr) or 56 hr post-transfection (24 hr) and were harvested at 80 hr post-transfection. Replicon RNA was measured by Taqman analysis of HCV RNA relative to the level of GAPDH mRNA in 0.5×10⁶ cells (Puig, M. et al. 2004 Vaccine 22:991-1000). Error bars represent ±SD. (D) Cells transfected with pcDNA3 vector (V) or E2 pcDNA3 (E2), were treated with 1,000 IU/mL IFN-α for 18 hr (+) and analyzed for PKR or actin expression by protein immunoblotting with polyclonal anti-PKR antiserum or anti-actin antibodies. (E) Immunoblot detection of phospho-eIF2α and total eIF2α (20 μg total protein/lane) from cells transfected with E2 or NS5A or E2 plus NS5A, and treated with 1,000 IU/mL IFN-α and poly I: poly C (20 μg/mL) for 18 hr (+).

FIG. 5 VA RNA_{I} rescues the replicon from the effects of IFN-α. (A) Luciferase activity in Huh.BB7 cells cotransfected with HCV IRES bicistronic reporter and pcDNA3, wild-type PKR (WT), PKR K296R, eIF2α S51A or VA RNA_{I}. Cells were treated with 500 IU/ml IFN-α for 18 hr. Luc activity is relative to the pcDNA3 control value, which was assigned a value of 1. (B) Taqman quantitation of HCV RNA from Huh.BB7 cells transfected with pcDNA3 (vector) or PKR inhibitors. Cells were untreated or treated with IFN-α at 500 IU/mL for 18 hr (+). The histogram shows the fold increase in RNA detected from an equal number of inhibitor-transfected cells relative to vector-transfected cells, which was assigned a value of 1, relative to GAPDH mRNA. Values are means ± standard deviation of the means (error bars). Below the histogram are the actual RNA copy numbers (10⁶) per reaction based on Taqman HCV RNA standards.

FIG. 6. IFN-α-treated Huh.BB7 cells yield edited replicon RNA. Four regions within the HCV IRES of BB7 replicon that contain mutations. The wild-type BB7 sequences are shown. The nucleotides in the wild-type BB7 sequence that were mutated are shown in bold type and underlined, and the nucleotides obtained after IFN-α treatment are shown below the sequence.

FIG. 7. A-to-I RNA editing in replicon-containing cells. (A) Thin layer chromatography of nuclease P1-digested RNA from Huh.BB7 cells grown in the presence of α-³²P [ATP] with (+) and without (−) 100 IU/mL IFN-α in the absence (−) and presence (+) of transfected VA RNA_{I} plasmid. Monophosphates were resolved on one TLC plate, although the rightmost lane was exposed longer. Radioactivity was quantitated by PhosphorImager analysis and shown below as a percentage of the total counts (IMP, ATP, AMP and origin). (B) Replicon-containing cells were transfected with VA RNA and treated with IFN-α (+). HCV replicon RNA was monitored by Taqman analysis and was quantitated relative to the amount of GAPDH RNA (Puig, M. et al. 2004 Vaccine 22:991-1000). Error bars represent standard deviation (±SD). Transfections were performed in duplicate with one dish of cells used for Taqman and the other used for immunoblot analysis (C) where cytoplasmic extracts (20 μg) were used to monitor ADAR1 and actin expression.

FIG. 8. siRNA knockdown of ADAR1 stimulates replicon expression. (A) Huh.BB7 cells were plated at 1×10⁶ and transfected once or twice with siRNA directed towards ADAR1 or siRNA directed against ADAR2. The histogram shows Taqman analysis of HCV RNA from equal numbers of cells. The number of transfections is shown below the bars in the histogram. (B) 25 μg of cytoplasmic lysates shown in panel A. siRNA experiments were repeated three times with similar results. (C) Huh.BB7 cells were transfected with ADAR1-specific siRNA and treated with IFN-α (10 or 1,000 IU/ml) at 4 days post-transfection. Cells were harvested after 18 hours of IFN-α treatment. RNA was isolated and monitored by Taqman analysis (Puig, M. et al. 2004 Vaccine 22:991-1000). (D) Cytoplasmic extracts were analyzed for protein expression as described in Examples. The migration positions of molecular mass markers are shown to the left of the blots.

### DETAILED DESCRIPTION OF THE PREFERRED EMBODIMENT

While many clinical hepatitis C virus infections are resistant to IFN-α (IFN-α) therapy, subgenomic in vitro self-replicating HCV RNA (HCV replicons), are characterized by marked IFN-α sensitivity. IFN-α treatment of replicon-containing cells results in a rapid loss of viral RNA via translation inhibition through double-stranded RNA-activated protein kinase (PKR) and also through a new pathway involving RNA editing by adenosine deaminase that acts on double-stranded RNA (ADAR1). More than 200 genes are induced by IFN-α and yet only a few are attributed with an antiviral role. We show that inhibition of PKR and ADAR1 by the addition of adenovirus-associated RNA stimulates replicon expression and reduces the amount of inosine recovered from RNA in replicon cells. Small inhibitory RNA, specific for ADAR1, stimulated the replicon 40-fold indicating that ADAR1 has a role in limiting replication of the viral RNA. This is the first report of ADAR's involvement in a potent antiviral pathway and its action to specifically eliminate HCV RNA through adenosine to inosine editing. These results help explain successful HCV replicon clearance by IFN-α in vitro and serve as the basis of a new therapeutic strategy for HCV as well as other viral infections.

Cell Culture Systems Permissive for HCV Infection and Growth

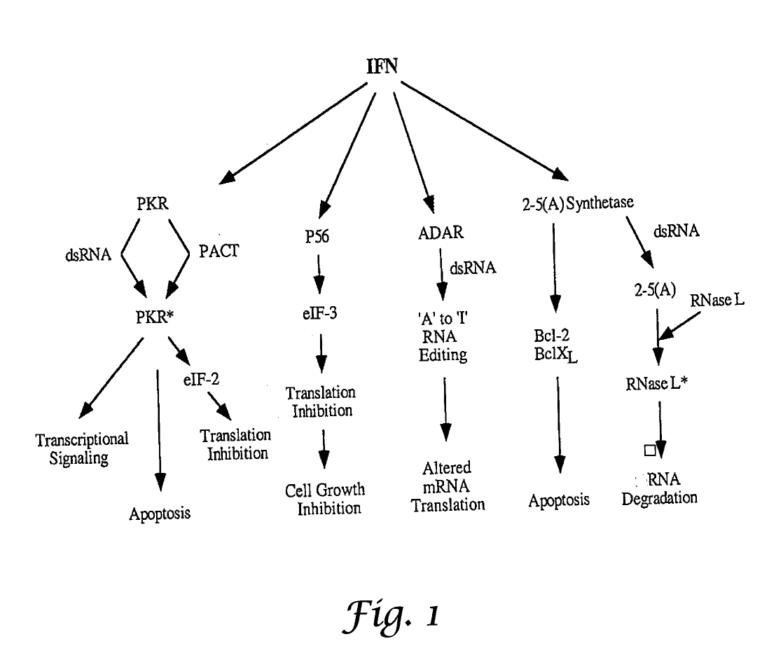

Several IFN-induced pathways affect cell growth, metabolism and the ability of RNA viruses to reproduce and propagate (FIG. 1). Among the many proteins induced by IFNs are PKR, P56, ADAR, and 2-5(A) synthetase. IFN-induced PKR is inactive as such and requires either ds RNA or another cellular protein, PACT, for its activation by autophosphorylation. Activated PKR (PKR*) is a protein kinase with stringent substrate specificity. It can phosphorylate eIF-2α to inhibit protein synthesis. It can also cause cellular apoptosis and participate in specific transcriptional signaling pathways. The IFN-induced protein, P56, binds to the P48 subunit of eIF-3 and inhibits its function, causing translation inhibition and cell growth inhibition. The IFN-induced ADAR adenosine deaminase edits (A to I) dsRNA structures in viral and cellular RNAs, thereby changing their coding properties. The IFN-induced 2-5 (A) synthetases are a family of enzymes, all of which are activated by dsRNA to synthesize 2′-5′-linked oligoadenylates [2-5(A)]. 2-5(A) binds and dimerizes inactive RNase L, causing its activation (RNaseL*), and activated RNaseL degrades RNA. A specific isozyme, 9-2 pf 2-5(A) synthetase has an additional cellular activity. It binds to the Bcl-2 family of proteins and causes cellular apoptosis. Suppression or elimination of the expression of interferon genes results in increased permissiveness of cells to viral replication.

The present invention identifies ADAR1 as a new pathway in the control of viral replication. Suppression or elimination of ADAR1 expression or activity significantly increases the permissiveness of interferon-deficient cells to viral replication. An increased permissiveness to viral reproduction means that greater viral production can be achieved relative to an interferon-deficient cell having normal ADAR1 expression. Cells having an increased permissiveness to viral replication are useful for a number of applications including vaccine production, sensitive detection of low levels of virus and for the evaluation of antiviral compounds.

Why do we need an infectious cell culture system?

We can study virus-host interactions (pathogenesis).

We can study interferon response (test new therapeutics for efficacy).

We can grow virus for vaccine (attenuated or inactivated).

We can assess either infectivity of implicated blood products or the effectiveness of viral inactivation procedures for the products.

The present inventors have surprisingly found that interferon-deficient animal cells that are also deficient in ADAR1 produce a higher viral yield when infected with an animal virus than interferon-deficient cells with normal levels of this protein. The ability to obtain high yields of virus in an interferon-deficient cell culture that is also deficient in ADAR1 makes it possible to produce large amounts of virus within a short time. This is particularly important for production of viral vaccines, most particularly for RNA viruses such as HCV. The increased permissiveness of the deficient cells to viral replication makes them useful in a method for evaluating antiviral drugs in cell culture and in a method for detecting viral pathogens.

On aspect of the present invention provides a method for production of a viral vaccine in cell culture which comprises (a) infecting a cell culture with a donor strain animal virus, wherein said cell culture is an interferon-deficient cell culture and is deficient in the activity of ADAR1, (b) culturing the infected cell culture under conditions sufficient to provide efficient virus growth, and (c) harvesting the virus produced. The harvested virus may be additionally prepared for vaccine use by purification, for instance by sterile filtration, ultrafiltration and/or concentration by column chromatography or other methods. The harvested virus may optionally be treated to inactivate the virus for the production of killed viral vaccines.

Several human leukemia cells and derived cell lines are known to be interferon-deficient due to homozygous deletion of their IFN-α and IFN-β genes (Diaz et al. 1988 Proc Natl Acad Sci USA 85:5259-5263). IFN-deficient cell lines include NALL-1 (Hiraki et al. 1977 Cancer 40:2131-2135), K562 (Lozzio C B and Lozzi B B 1975 Blood 45:321-334), R24;11 (Stong R C et al. 1985 Blood 65:21-31), Reh (Rosenfeld C et al. 1977 Nature 267:841-843), SUP-T3 and K-T1 (Smith S D et al. 1986 Blood 67:650-656) and Vero cells (American Public Health Association). Compendium of methods for the microbiological examination of foods. 3rd ed. Washington, D.C.: American Public Health Association; 1992). In addition to IFN-deficient cell lines that are already available, other cell lines of choice may be modified by recombinant methods that are well known in the art to become IFN-deficient through inactivation or deletion of IFN-α and IFN-β genes. It is readily apparent to those of ordinary skill in the art that homologs of human IFN-α and -β genes can be inactivated or deleted in cells of nonhuman origin.

In a preferred embodiment, the interferon-deficient cell culture is deficient in ADAR1 activity. By ADAR1-deficient is meant that the ADAR1 activity is less than 50%, 40%, 30%, 25%, 20%, 15%, 10%, or 5% of the normal level of ADAR1 activity. By normal level of ADAR1 activity is meant the ADAR activity observed in the parental cell culture from which the stable ADAR1-deficient cells are obtained or, if the ADAR1-deficiency is transiently induced, the ADAR1 activity level observed in the cells before induction to ADAR1-deficiency. Preferably, the ADAR1-deficient cells have less than 5%, 4%, 3%, 2%, or 1% of the normal level of ADAR1 activity, more preferably the ADAR1-deficient cells have less than 0.1% of the normal level of ADAR1 activity. By ADAR1 activity is meant the ability to mediate the antiviral and antiproliferative activities of IFN-α and IFN-β, the ability to edit double stranded RNA and to catalyze the deamination of adenosine residues resulting in inosine substitution. By ADAR1 is meant human ADAR1 or any analog or homolog of human ADAR1. By analog of human ADAR1 is meant any double-stranded RNA-dependent adenosine deaminase that mediates ds-RNA RNA editing, having at least one dsRNA binding protein (DRBP) domain and at least one deaminase domain. By homolog is meant a protein homologous to at least one domain of ADAR1, such as, for example, the dsRNA-binding protein domain or the deaminase domain. Typically, such ds-RNA dependent double-stranded RNA adenosine deaminases are present in other species such as rats, Xenopus laevis, C. elegans, etc., and in different tissues among the various species. For example, rat ADAR1 is an analog of human ADAR1. Another analog of ADAR1 is Xenopus laevis ADAR1. Still another analog of ADAR1 is C. elegans ADAR1. A functional ADAR1 homolog is ADAR2, another is ADAR3, and still another is Testis nuclear RNA binding protein (TENR).

ADAR1 -deficient cells can be obtained by any of a variety of methods that are well-known in the art. ADAR1-deficient mutants can be stably ADAR1-deficient or may be transiently induced to ADAR1-deficiency. Techniques for producing stable ADAR1-deficient mutants include, but are not limited to, random or site-directed mutagenesis (for example, Deng W P and Nickoloff J A 1992 Analytical Biochemistry 200:81-88; Busby S et al. 1982 Mol Biol 154:197-209), targeted gene deletion (“gene knock-out”) (for example, Camper S A, et al. 1995 Biology of Reproduction 52:246-257; Aguzzi A et al. 1994 Brain Pathology 4:3-20), transfection with ADAR1 antisense polynucleotides, RNA interference (Jayan, G C and Casey, J L 2002 J Virol 76:12399-12404), transfection with adenovirus associated RNA (VA RNA), transfection with genes encoding peptides (such as, for example peptides representing those regions of ADAR1 required for its binding to dsRNA or its RNA editing), treatment with small organic or inorganic molecules that, for example, target the catalytic domain or active site and that may resemble nucleosides or nucleoside analogs, transfection with genes encoding antibodies (including, for example, polyclonal, monoclonal, humanized, anti-idiotypic, chimeric or single chain antibodies, and FAb, F(ab′)2 and FAb expression library fragments, and epitope-binding fragments thereof), transfection with ribozymes, transfection with triple helix polynucleotides, transfection with genes encoding proteins that inhibit ADAR, and transfection with an ADAR1 dominant negative mutant gene.

An ADAR1 dominant mutant is an ADAR1 mutant for which only a single allele need be expressed in order to suppress normal ADAR1 activity. ADAR1 dominant mutant genes include a mutant human ADAR1, a mutant rat ADAR1, a mutant Xenopus laevis ADAR1, a mutant C. elegans ADAR1, and mutants of any other adenosine deaminases that act on double-stranded RNA or mutants of analogs or homologs of human ADAR1 that suppress normal ADAR1 activity. Examples of other ADAR1 dominant mutants include mutated, truncated, or deleted forms of ADAR1. ADAR1 dominant mutants include mutants of functional homologs that suppress adenosine deamination or that block binding to double-stranded RNA.

In some embodiments, cell cultures will be stably ADAR1-deficient, and in other embodiments, cell cultures will be transiently ADAR1-deficient. Typically, ADAR1-deficient cell cultures are produced by transfection of an interferon-deficient parental cell line, preferably a cell line currently used in vaccine production, preferably Vero (African Green Monkey cell), with a small inhibitory RNA (siRNA) specifically directed to knockdown the expression of ADAR1 or a vector containing a functional ADAR1 antisense gene construct or an ADAR1 dominant negative mutant construct followed by selection of those cells that have received the vector. A small inhibitory RNA (siRNA) may be prepared by conventional methods, for example, by preparing a siRNA such as that described in Jayan, G C and Casey, J L 2002 J Virol 76:12399-12404. A functional ADAR1 antisense gene construct may be prepared by conventional methods, for example, by cloning an ADAR1 cDNA in an antisense orientation, under the control of an appropriate promoter. An ADAR1 dominant negative mutant construct can be prepared by cloning the cDNA for an ADAR1 dominant negative mutant under the control of an appropriate promoter.

Various ways of constructing different vectors, for example using chemically or enzymatically synthesized DNA, fragments of the ADAR1 cDNA or ADAR1 gene, will be readily apparent to those skilled in the art. Transfection of the parental cell culture is carried out by standard methods, for example, the DEAE-dextran method (McCutchen and Pagano 1968 J Natl Cancer Inst 41:351-357), the calcium phosphate procedure (Graham and van der Eb 1973 Virology 52:456-467) or by any other method known in the art, including but not limited to microinjection, lipofection, and electroporation. Such methods are generally described in Sambrook et al., Molecular Cloning: A laboratory manual, 2nd Edition, 1989, Cold Spring Harbor Laboratory Press. Transfectants having deficient ADAR1 activity are selected. For ease of selection, a marker gene such as neomycin phosphotransferase II, ampicillin resistance or G418 resistance, may be included in the vector carrying the antisense or mutant gene. When a marker gene is included, the transfectant may be selected for expression of the marker gene (e.g. antibiotic resistance), cultured and then assayed for ADAR1 activity.

Residual ADAR1 activity in ADAR1-deficient cells can be determined by any of a number of techniques that are well-known in the art. The activity of ADAR1 can be determined directly by, for example, by measuring conversion of adenosine of dsRNA to inosine such as described in Kim et al. 1994 J Biol Chem 269:13480-13489 or RNA editing in replicon-containing cells such as described in Jayan, G C and Casey, J L 2002 J Virol 76:12399-12404.

Residual ADAR1 activity may also be determined indirectly by assaying for the presence of the ADAR1 protein, for example by Western blot with ADAR1 specific antibodies, or for the presence of ADAR1 RNA, for example by Northern blot with oligonucleotide or cDNA probes specific for ADAR1.

As will be readily apparent, the type of assay appropriate for determination of residual ADAR1 activity will in most cases depend on the method used to obtain the ADAR1-deficient phenotype. If, for example, the method used to produce the ADAR1-deficient cell results in suppression or elimination of ADAR1 gene expression (for example, gene knock-out), analysis techniques that detect the presence of mRNA or cDNA (e.g. Northern or Southern blots) or the presence of the protein (e.g. Western blot) or that detect the protein activity may be useful to determine the residual ADAR1 activity in the ADAR1-deficient cells. On the other hand, if the method used to produce the ADAR1-deficient cells results in inhibition of the protein rather than elimination of expression of the gene (for instance, transfection with a vector carrying a dominant negative ADAR1 mutant), a functional ADAR1 assay is more appropriate than a Western blot for determination of the residual ADAR1 activity.

In another embodiment, the present invention provides a method for production of a viral vaccine in a cell culture that is deficient in PKR activity. A cell culture deficient in PKR can be isolated in a similar fashion to cell cultures deficient in ADAR1, for example, random or site-directed mutagenesis, targeted gene deletion of the PKR genes or transfection with antisense PKR constructs. By PKR-deficient is meant that the PKR activity is less than 5% of the normal level of PKR activity. By normal level of PKR activity is meant the PKR activity observed in the parental cell culture from which the stable PKR-deficient cells are obtained or, if the PKR-deficiency is transiently induced, the PKR activity level observed in the cells before induction to PKR-deficiency. Preferably, the PKR-deficient cells have less than 1% of the normal level of PKR activity, more preferably the PKR-deficient cells have less than 0.1% of the normal level of PKR activity. Residual PKR activity in PKR-deficient cells can be determined by methods similar to those used for determining residual ADAR1 activity, that is, Western blots using PKR specific antibodies, Northern blots using oligonucleotide or cDNA probes specific for PKR or enzyme activity assays, for example, by testing for autophosphorylation such as described in Maran et al. (1994 Science 265:789-792) or Silverman et al. (Silverman, R. H., and Krause, D. (1986) in Interferons: A practical approach. Morris, A. G. and Clemens, M. J., eds. pp. 71-74 IRL Press, Oxford-Washington, D.C.).

In another embodiment, the present invention provides a method for production of a viral vaccine in a cell culture that is deficient in 2′-5′ oligoadenylate synthetase activity. A cell culture deficient in 2-5A synthetase can be isolated in a similar fashion to cell cultures deficient in ADAR1 and PKR, for example, random or site-directed mutagenesis, targeted gene deletion of the 2-5A synthetase genes or transfection with antisense 2-5A synthetase constructs. By 2-5A synthetase-deficient is meant that the 2-5 A synthetase activity is less than 5% of the normal level of 2-5A synthetase activity. By normal level of 2-5A synthetase activity is meant the 2-5A synthetase activity observed in the parental cell culture from which the stable 2-5A synthetase-deficient cells are obtained or, if the 2-5A synthetase-deficiency is transiently induced, the 2-5A synthetase activity level observed in the cells before induction to 2-5A synthetase-deficiency. Preferably, the 2-5A synthetase-deficient cells have less than 1% of the normal level of 2-5A synthetase activity, more preferably the 2-5A synthetase-deficient cells have less than 0.1% of the normal level of 2-5A synthetase activity. Residual 2-5A synthetase activity in 2-5A synthetase-deficient cells can be determined by methods similar to those used for determining residual PKR activity, that is, Western blots using 2-5A synthetase specific antibodies, Northern blots using oligonucleotide or cDNA probes specific for 2-5A synthetase or enzyme activity assays (Read et al. 1985 J Infect Dis 152:466-472; Hassel and Ts'o 1994 J Virol Methods 50:323-334).

In another embodiment, the present invention provides a method for production of a viral vaccine in a cell culture that is deficient in human Mx protein activity. The Mx proteins are induced specifically by IFN-α/β (Staeheli P 1990 Adv Virus Res 38:147-200). They are large GTPases that belong to the dynamin superfamily (Horisberger M A 1992 J Virol 66:4705-4709). The Mx proteins are known to exert an antiviral effect by targeting specific steps of the viral replication cycle of RNA viruses (Haller et al. 1998 Rev Sci Tech 17:220-230). A cell culture deficient in human Mx protein activity can be isolated in a similar fashion to cell cultures deficient in PKR, for example, random or site-directed mutagenesis, targeted gene deletion of the Mx genes or transfection with antisense Mx constructs. By Mx protein-deficient is meant that the Mx activity is less than 5% of the normal level of Mx activity. By normal level of Mx activity is meant the Mx activity observed in the parental cell culture from which the stable Mx-deficient cells are obtained or, if the Mx-deficiency is transiently induced, the Mx activity level observed in the cells before induction to Mx-deficiency. Preferably, the Mx-deficient cells have less than 1% of the normal level of Mx activity, more preferably the Mx-deficient cells have less than 0.1% of the normal level of Mx activity. Residual Mx activity in Mx-deficient cells can be determined by methods similar to those used for determining residual PKR activity, that is, Western blots using Mx specific antibodies, Northern blots using oligonucleotide or cDNA probes specific for Mx or enzyme activity assays (Garber et al. 1991 Virology 180:754-762; Zurcher et al. 1992 J Virol 66:5059-5066).

It will be apparent that cell cultures deficient in ADAR1 activity, PKR activity, 2-5A synthetase activity and Mx activity can be made by a combination of the methods described above. The multiply deficient cell cultures can be prepared either sequentially (that is, by first selecting cultures deficient in one activity and then using that cell culture as the starting material for preparing the second deficient culture) or simultaneously (selection for multiple deficiencies at once).

The method of the present invention can be practiced with a variety of animal cell cultures, including primary cell cultures, diploid cell cultures and continuous cell cultures. Particularly useful are cell cultures that are currently used for the production of vaccines, most particularly those cell cultures that have been approved for vaccine production by the USFDA and or WHO, for example, MRC-5, a human diploid cell line from fetal lung tissue (Jacobs et al. 1970 Nature 227:168-170) and WI-38, a human diploid cell line derived from embryonic lung tissue (Hayflick and Moorhead 1961• Exp Cell Res 25:585-621). Also useful are Chang liver cells (Chang 1954 Proc Soc Exp Biol Med 87:440), U937 human promonocytic cells (Sundstrom and Nilsson 1976 Int J Cancer 17:565-577), Vero cells, MRC-9 cells, IMR-90 cells, IMR-91 cells, Lederle 130 cells and Huh7 cells. For a general review of cell cultures used in the production of vaccines see Grachev, V. P. in Viral Vaccines Mizrahi, A. ed. pages 37-67 1990 Wiley-Liss. The particular cell culture chosen will depend on the virus which is to be produced; in general, the cell culture will be derived from the species which is the natural host for the virus, although this is not essential for the practice of the present invention (for example, human virus can be grown on a canine kidney cell line (MDCK cells) or a green monkey kidney cell line (Vero cells; Swanson et al. 1988 J Biol Stand 16:311)). Typically, the cells chosen will be interferon-deficient cells or cell lines known to be an appropriate host for the virus to be produced. Cell lines used for the production of vaccines are well known and readily available from commercial suppliers, for example, American Type Culture Collection.

The infection of the interferon-deficient cells that are also deficient in ADAR1 with donor virus according to the present invention is carried out by conventional techniques (see for example Peetermans 1992 J Vaccine 1O:S99-101). Typically, virus is added to the cell culture at between 0.001 to 0.5 TCID₅₀ per cell, preferably at 0.01 to 0.10 TCID₅₀ per cell, but will vary as appropriate for the particular virus and cell host being used. As is readily apparent to one of ordinary skill in the art, every cell of the cell culture need not be infected initially for efficient viral production. The infected cells are cultured under conditions appropriate for the particular cells and viral production at various times after infection is monitored. Viral production can be monitored by any of a number of standard techniques including plaque-forming unit assays, TCID₅₀ assays or hemagglutination inhibition assays (Robertson et al. 1991 J Gen Virol 72:2671-2677). The infected cells are cultured under conditions sufficient to provide efficient viral growth. The cells can be cultured until maximum viral production is achieved as indicated by a plateauing of the viral yield. The virus is harvested by standard techniques and substantially purified from other cellular components (see for example, Peetermans 1992 J Vaccine 10:S99-101). The harvested virus may be used as a live viral vaccine, either fully virulent or attenuated, or may be inactivated before use by methods that are well-known in the art, for example, by treatment with formaldehyde (Peetermans 1992 J Vaccine 10:S99-101).

The vaccine may be available in dry form, to be mixed with a diluent, or may be in liquid form, preferably in aqueous solution, either concentrated or ready to use. The vaccine is administered alone or in combination with pharmaceutically acceptable carriers, adjuvants, preservatives, diluents and other additives useful to enhance immunogenicity or aid in administration or storage as are well-known in the art. Suitable adjuvants include aluminum hydroxide, alum, aluminum phosphate and Freund's adjuvant. Other suitable additives include sucrose, dextrose, lactose, and other non-toxic substances. The vaccines are administered to animals by various routes, including intramuscular, intravenous, subcutaneous, intratracheal, intranasal, or by aerosol spray and the vaccines are contemplated for the beneficial use in a variety of animals including human, equine, avian, feline, canine and bovine.

The method of the present invention can be practiced with a variety of donor animal viruses. By donor virus is meant the particular viral strain that is replicated in vitro to produce the vaccine. The particular donor animal virus used will depend upon the viral vaccine desired. Donor viruses currently used for vaccine production are well-known in the art and the method of the present invention can be readily adapted to any newly identified donor virus. Preferred donor viruses include positive-sense ss RNA viruses, including members of the family Arteriviridae, Astroviridae, Caliciviridae, Coronaviridae, Flaviviridae, Picomaviridae, and Togaviridae; negative-sense ss RNA viruses, including members of the family Arenaviridae, Bornaviridae, Bunyaviridae, Filoviridae, Orthomyxoviridae, Paramyxoviridae, and Rhabdoviridae; dsRNA viruses, including members of the family Reoviridae; and reverse-transcribing viruses, including members of the family Retroviridae, but donor viruses may also include DNA viruses, including members of the family Adenoviridae, Herpesviridae, Papillomaviridae, Polyomaviridae, Poxviridae, and Parvoviridae The donor virus can be identical to the viral pathogen or may be a naturally-occurring attenuated form, an attenuated form produced by serial passage through cell culture or a recombinant form. Any viral strain may be used as donor virus provided that it retains the requisite antigenicity to afford protection against the viral pathogen. The method of the present invention is particularly useful with attenuated or poorly replicating donor viruses.

Some of the vaccines that can be provided by the methods of the present invention include, but are not limited to, human vaccines for poliovirus, measles, mumps, rubella, hepatitis A, influenza, parainfluenza, Japanese encephalitis, cytomegalovirus, HIV, Dengue fever virus, rabies, Varicella-zoster virus and hepatitis C, as well as many non-human animal vaccines including, for example, vaccines for feline leukemia virus, bovine rhinotracheitis virus (red nose virus), cowpox virus, canine hepatitis virus, canine distemper virus, equine rhinovirus, equine influenza virus, equine pneumonia virus, equine infectious anemia virus, equine encephalitis virus, ovine encephalitis virus, ovine blue tongue virus, rabies virus, swine influenza virus and simian immunodeficiency virus. As will be apparent from the foregoing, the method of the present invention is not limited to vaccine production for human viruses but is equally suitable for production of non-human animal viral vaccines.

Another aspect of the present invention provides a method for evaluating the activity of antiviral compounds. Examples of compounds include experimental drugs, antibodies, small organic or inorganic molecules, etc. Due to the increased permissiveness of the interferon-deficient cells that are also deficient in ADAR1 to viral replication, the cells are useful in a sensitive assay for assessing the effectiveness of antiviral compounds. In this aspect, the present invention comprises the steps of (a) treating a virus, virus-infected host cells or host cells prior to virus infection with the antiviral compound and (b) assaying for the presence of remaining infectious virus by exposure under infective conditions of an interferon-deficient indicator cell culture that is also deficient in ADAR1.

In this aspect, the virus against which the antiviral compound is to be tested may be treated directly with the compound. In this case, the treated virus may then be analyzed directly for the presence of remaining infectious virus by exposure under infective conditions of an interferon-deficient indicator cell culture that is also deficient in ADAR1 to an aliquot of the treated virus, culturing for a time sufficient to allow replication of any remaining infectious virus and analyzing the indicator culture for the presence of the replicated virus. Alternatively, the virus against which the antiviral compound is to be tested may be used to infect a host cell culture; the infected host cell culture is then treated with the antiviral compound. A cell extract of the treated infected host cell culture is prepared by conventional techniques and an aliquot of the extract is analyzed for the presence of remaining infectious virus by exposure to an interferon-deficient indicator cell culture that is also deficient in ADAR1 as described above. In another alternative, the host cell culture may be treated with the antiviral compound prior to infection with the virus rather than after infection. The treated cells are then infected with the virus against which the antiviral compound is to be tested, cultured and analyzed for the presence of replicated virus. The particular treatment regime chosen will depend upon the known or postulated mode of action of the antiviral compound and will be readily within the determination of one skilled in the art. By exposure under infective conditions is intended the bringing together the deficient indicator cell culture and an aliquot of the treated sample (either virus or infected cell extract) under conditions that would result in infection of the deficient cell culture if any virus was present in the treated sample. After exposure to the treated sample, the deficient indicator cell culture is cultured further and assayed for the replication of the virus, by standard methods (for example, plaque assays or TCID₅₀ assays or Northern or Western analysis for viral RNA or protein).

The host cell culture may be any cell culture which is susceptible to infection by the virus against which the antiviral compound is to be tested. The indicator cell culture is an interferon-deficient cell culture that is also deficient in ADAR1 and that is used to assay for infectious virus remaining after treatment with the antiviral compound. The indicator interferon-deficient cell culture that is also deficient in ADAR1 is prepared as described above for vaccine production. Cells suitable as a parent for generating the deficient indicator are the same as those that are useful for generating the interferon-deficient cell cultures that is also deficient in ADAR1 for vaccine production. In addition, the following cell lines are also suitable: hepatoma cell lines in general, particularly Hep G2 human hepatocellular carcinoma (Aden D P et al. 1979 Nature 282:615-616), Hep 3B and Huh7. It will be apparent that the indicator cell culture is also susceptible to infection by the virus against which the antiviral compound is to be treated. The host cell culture and the indicator cell culture may be the same or different. The antiviral compound can be any chemical or biological preparation suspected of having some antiviral activity. If the virus itself is treated with the antiviral compound, the antiviral compound may be removed before infection of the indicator cell culture by exposure to the treated virus. If an infected host cell culture (or a pre-infected host cell culture) is treated with the antiviral compound, the compound may be removed before preparation of the cell extract.

In a separate related aspect, the present invention provides a method for identification and culture of viral pathogens. The permissiveness of interferon-deficient cells that are also deficient in ADAR1 to viral replication makes them particularly useful in a method to detect very low levels of virus and/or viruses that are difficult to culture, for example, HIV in monocytes or lymphocytes of neonates. In this aspect the present invention comprises the steps of (1) exposing under infective conditions an interferon-deficient cell culture that is also deficient in ADAR1 to a sample suspected of containing a virus and (2) assaying for the presence of replicated virus in the exposed cells. The practice of this aspect of the present invention is similar to that of the previous aspect except that treatment with antiviral compound is omitted. In this aspect, the sample to be assayed for the presence of virus is generally a clinical sample from a patient suspected of having a viral infection. The sample may be any appropriate clinical sample including blood, saliva, urine, as well as biopsy samples from lymph node, lung, intestine, liver, kidney and brain tissue. The sample may be treated appropriately to release viral particles (for example, cell extracts may be prepared) or the sample may be used as received from the patient. The sample or an aliquot of the sample is exposed under infective conditions to a deficient indicator cell culture and the presence of any replicating virus is determined as described above.

### Combination Antiviral Therapy

Although an interferon-α treatment for chronic hepatitis C virus (HCV) infection is widely available, sustained responses—ie, those that persist into the follow-up period—are found in only 10-20% of patients. The response rate may increase on high-dose regimens or with long-term treatment, but there is a concomitant increase in side-effects and cost of treatment, and decrease in patient compliance. New treatment regimens are needed.

Ribavirin (1-D-ribofuranosyl-1H-1,2,4-triazole-3-carboxamide) is a nucleoside analogue that inhibits the replication of many different RNA and DNA viruses, including some related to HCV. Oral ribavirin treatment of chronic HCV infection is generally well tolerated but although a biochemical response occurs in most patients, the effect on HCV replication is small.

Some studies found that the combination of an interferon-α and ribavirin increased the sustained-response rate compared with the rates seen with either drug alone. Reichard et al. (The Lancet 1998; 351:83-87) carried out a randomized, double-blind, placebo-controlled study to compare the efficacy and safety of the combination of an interferon-α and ribavirin with the interferon-α alone.

In this double-blind trial 100 patients were randomly assigned to treatment with a interferon-α (3 MU three times a week) in combination with ribavirin (1000 or 1200 mg per day) or placebo for 24 weeks and then followed up for a further 24 weeks. A further follow-up was done 1 year after active treatment stopped. The primary endpoint was the sustained virological response, defined as no detectable HCV RNA by PCR at both week 24 and week 48. Retrospectively, the baseline HCV-RNA load was analyzed

More patients with chronic hepatitis C have a sustained virological response with the interferon-a and ribavirin than with only the interferon-α treatment.

Other combinations with interferon are needed. Accordingly, an embodiment of the present invention provides the combination of an ADAR1 agonist and an interferon-α for treating patients having infection by hepatitis C virus or another related virus. Still another embodiment of the present invention provides a method of treating patients having infection with hepatitis C or another related virus comprising administering a therapeutically effective amount of an interferon-α and a therapeutically effective amount of an ADAR1 agonist for a time sufficient to eradicate detectable virus for at least 24 weeks after the end of said period of administrating.

The treating of patients with the combination is performed as part of a combination therapy with an, interferon-α, including interferon α-2a, interferon α-2b, consensus interferon, as well as pegylated interferon α-2a and pegylated interferon α-2b. Pegylated interferon-α formulations are not effective when administered orally, so the preferred method of administering the pegylated interferon-α is parenterally, preferably by sub-cutaneous (SC), intravenous (IV), or intramuscular (IM) injection.

The treatment does not exclude ribavirin or its derivatives. These may be administered orally in capsule, tablet, or liquid form, intranasally as an aerosol by nasal spray, or parenterally, preferably by SC, IV, or IM injection.

Of course, other types of administration of both medicaments, as they become available, are contemplated, such as transdermally, by suppository, by sustained release dosage form, and by pulmonary inhalation. Any form of administration will work so long as the proper dosages are delivered without destroying the active ingredient.

The term “an interferon-α” as used herein means the family of highly homologous species-specific proteins that inhibit viral replication and cellular proliferation and modulate immune response. The use of interferon α-2a or α-2b is preferred.

The term “pegylated interferon” as used herein means polyethylene glycol modified conjugates of interferon.

Pharmaceutical composition of interferon, suitable for parenteral administration may be formulated with a suitable buffer, e.g., Tris-HCI, acetate or phosphate such as dibasic sodium phosphate/monobasic sodium phosphate buffer, and pharmaceutically acceptable excipients (e.g., sucrose), carriers (e.g. human serum albumin), tonicity agents (e.g. NaCl), preservatives (e.g. thimerosol, cresol or benylalcohol), and surfactants (e.g. Tween or polysorbates) in sterile water for injection. The interferon may be stored as lyophilized powders under refrigeration at 2-8° C. The reconstituted aqueous solutions are stable when stored between 2-8° C. and used within 24 hours of reconstitution. The reconstituted aqueous solutions may also be stored in pre-filled, multi-dose syringes such as those useful for delivery of insulin. Other syringe systems include a syringe comprising a cartridge containing a diluent and lyophilized interferon powder in a separate compartment.

The notion of no detectable virus in the context of the present invention means fewer copies than the lower level of detection.

The invention also provides a method for screening a compound for effectiveness as an agonist of ADAR1. The method comprises a) exposing a sample comprising ADAR1 to a compound, and b) detecting agonist activity in the sample. To identify agonists, an upregulation of ADAR RNA or protein and/or editing activity could be observed. The presence or absence of inosine monophosphate in cells that disappears with the knockdown of ADAR expression is consistent with an ADAR agonist. As described above, the invention provides a composition comprising an agonist of ADAR1. In another alternative as described above, the invention provides a method of treatment comprising administering to a patient in need of such treatment the agonist of ADAR1.

### A New Antiviral Pathway that Mediates HCV Replicon Interferon Sensitivity through ADAR1

IFN-α Sensitivity in Cell Culture is Replicon Specific

The mechanisms underlying IFN-α resistance by HCV have been examined with the replicon-based system (Lohmann, V et al. 1999 Science 285:110-113). Efficient replication of HCV RNA was observed in Huh7 cells that had been stably transfected with the HCV replicon (BB7; Lohmann, V et al. 1999 Science 285:110-113; Blight, K J et al. 2000 Science 290:1972-1974; Blight, K J et al. 2002 J Virol 76:13001-13014). While the replicons were derived from a genotype 1 b strain, which is usually the most interferon-resistant, these replicons were highly sensitive to IFN-α (Blight, K J et al. 2000 Science 290:1972-1974). It was even possible to completely cure the cells of the replicon with IFN-α treatment (Blight, K J et al. 2002 J Virol 76:13001-13014). In addition, sequence adaptations that conferred robust growth in cell culture did not reduce IFN-α sensitivity (Blight, K J et al. 2000 Science 290:1972-1974).

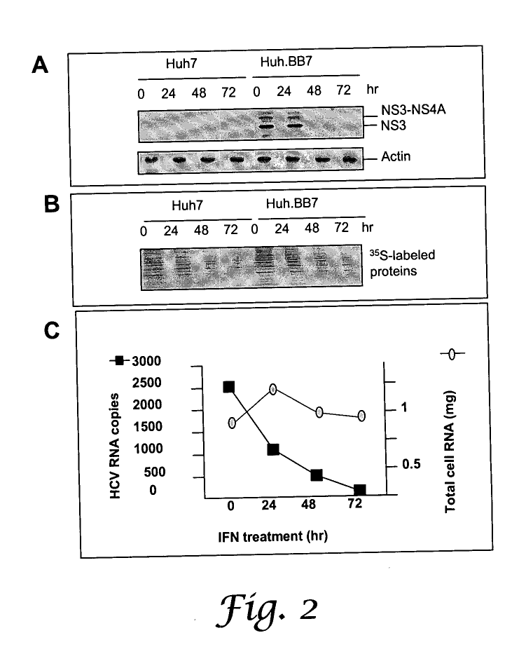

We found that expression of the HCV replicon proteins was highly sensitive to IFN-α (FIG. 2A). Total protein synthesis in Huh7 or replicon-containing cells (Huh.BB7) was also affected as demonstrated by the decrease in total cellular proteins synthesized after IFN-α treatment (FIG. 2B). IFN-α treatment caused relatively little cytotoxicity in cells that contained the replicon or in the parental cells (Huh7). We performed Northern blot analysis to detect replicon RNA in IFN-α-treated cells. With increasing levels of IFN-α, a decrease in replicon RNA was observed. As previously reported by Blight et al. (Blight, K J et al. 2000 Science 290:1972-1974), we observed a precipitous decrease in replicon RNA after IFN-α treatment as measured by Taqman analysis, while total RNA in the cells was largely unaffected (FIG. 2C), demonstrating that the primary effects of IFN-α are replicon specific.

The HCV IRES is IFN-α Resistant in Replicon-Containing Cells

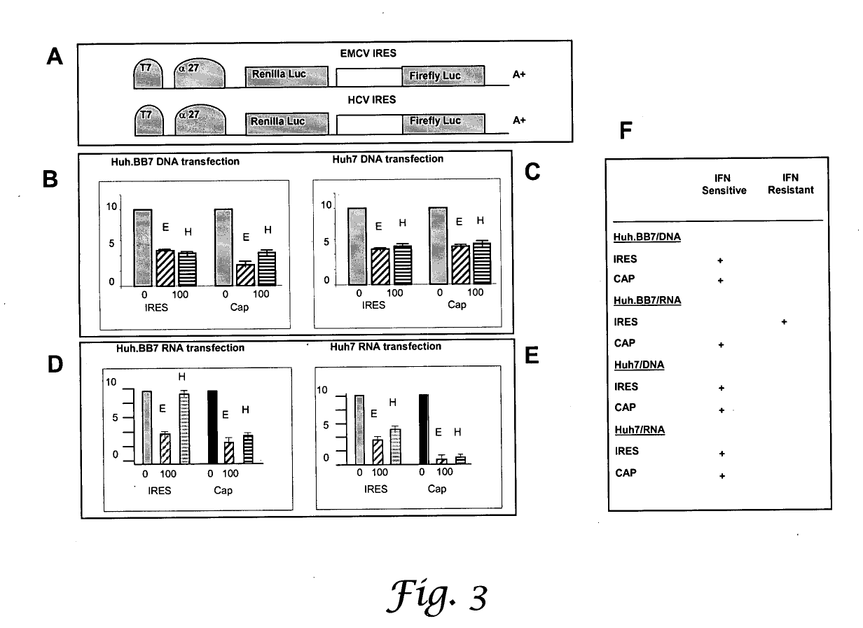

Translation of HCV is initiated at the highly structured 5′ untranslated region of the viral RNA containing an internal ribosome entry site (IRES). To determine how replicon-specific protein synthesis was affected by IFN-α, we engineered a bicistronic DNA reporter that expresses Renilla luciferase (Luc) under a 5′ cap-dependent translational mechanism and firefly Luc under a cap-independent, IRES-directed mechanism. We examined the effects of IFN-α on expression from both the encephalomyocarditis virus (EMCV) IRES and the HCV IRES (FIG. 3A), because both are required for replicon expression. Both cap-dependent and EMCV IRES-dependent luc expression was inhibited by IFN-α in both Huh7.BB7 and Huh7 cells (FIG. 3B, C). Complete inhibition of Luc activity was not observed, because Luc protein accumulated for 30 hours prior to the addition of IFN-α. HCV IRES-dependent Luc expression was also inhibited by IFN-α (FIG. 3C), indicating that there is a global effect of IFN-α at the post-transcriptional stage in transfected cells. This is consistent with an inhibition of translation by PKR, as PKR activation would result in a decrease in both cap- and IRES-dependent translation.

To exclude the possibility that transcription of the Luc reporter was inhibited by IFN-α, we compared Luc expression using transfected DNA to Luc expression from transfected capped RNA synthesized in vitro from the same reporter and observed radically different responses to IFN-α treatment. When DNA was transfected into Huh.BB7 cells, the EMCV-IRES, HCV-IRES and cap-dependent expression of Luc were all inhibited by IFN-α (FIG. 3B). The parental Huh7 cells lacking the replicon behaved similarly (FIG. 3C). However, when we transfected RNA encoding the Lucreporter, only HCV IRES-dependent Luc expression was resistant to IFN-α in those cells that contained the replicon (FIG. 3D) and was sensitive to IFN-α in Huh7 cells (FIG. 3E). These results were repeated with similar reporters described previously and are consistent with those reported by Koev et al. who proposed that a PKR-independent mechanism is responsible for IFN-α activity on the replicon (Koev, G et al. 2002 Virology 297:195-202). Because PKR activation results in inhibition of both cap-dependent and -independent translation, if PKR were solely responsible for IFN-α sensitivity of the cap-dependent Luc expression, then IRES-dependent Luc expression should be affected as well. These results show that in transfected cells, in the absence of the replicon, both cap-dependent and -independent translation were inhibited (FIG. 3C, E), consistent with inhibition due to activated PKR, but does not exclude other mechanisms of inhibition. They are contradictory to results demonstrating that the replicon is sensitive to G418 in the presence of IFN-α. Therefore, the HCV IRES, in the context of the replicon, is sensitive to IFN-α. These data indicate that the RNA of the HCV-IRES-containing reporter, in the presence of the replicon may be inhibiting or blocking the main effects of IFN-α on IRES translation in a manner that cannot be fulfilled by the DNA reporter or in cells that lack the replicon (FIG. 3F). Because PKR is activated by dsRNA, which is probably present in replicon-containing cells, translation inhibition through PKR is expected. However, PKR did not shut down translation of the HCV IRES reporter in replicon cells. Taken together, it appears that while PKR may be activated, this cannot fully explain the IFN-α sensitivity of the replicon.

E2 and NS5A Stimulate Translation but did not Rescue the replicon from IFN-α Sensitivity

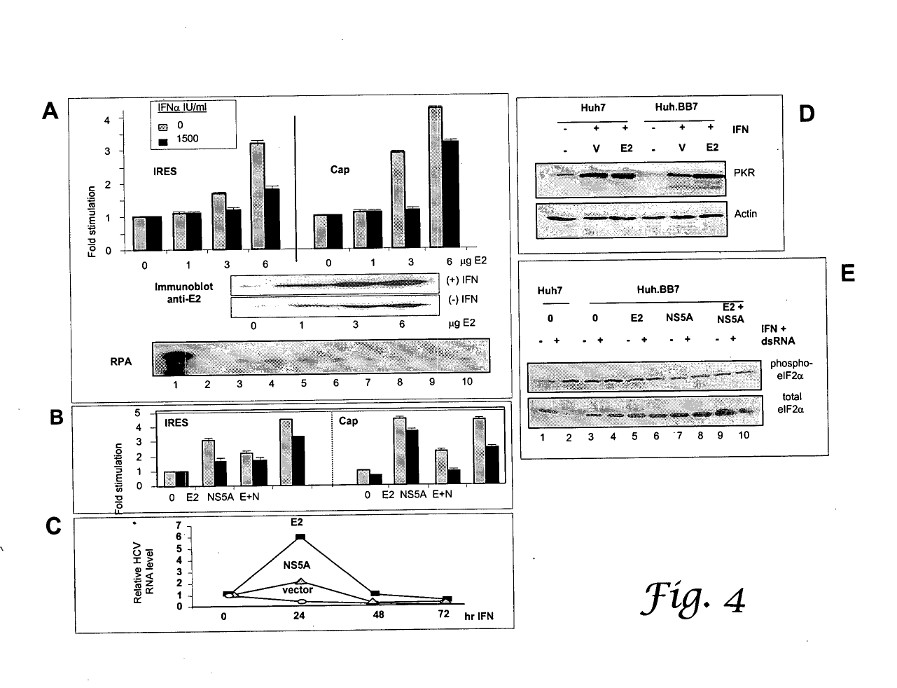

To examine the importance of PKR-dependent translation inhibition on the replicon, we used PKR inhibitors to counteract the effect of IFN-α in the Luc assay. Increasing amounts of transfected E2 stimulated both cap-dependent and IRES-dependent translation with and without IFN-α (FIG. 4A). Stimulation of translation corresponded with increasing levels of E2 from cells cotransfected with the Luc reporter. Equal amounts of Luc reporter were transfected in these cells, as demonstrated by the RNase protection assay, below (FIG. 4A, RPA). Both E2 and NS5A have been shown to inhibit PKR (Saunders, L R and Barber, G N 2003 FASEB J 17:961-983; Scadden, A D J and Smith, C W J 1997 EMBO J 16:2140-2149) and expression of each of these HCV genes enhanced Luc expression in the presence and absence of exogenous IFN-α (FIG. 4B). We used a high level of IFN-α to show the enhanced affect of E2 and NS5A. No differences in reporter expression were seen in Huh7 cells transfected with vector DNA. Interestingly, the combination of both E2 and NS5A enhanced Luc expression in Huh.BB7 cells in an additive manner (FIG. 4B), indicating that E2 and NS5A counteract the effects of IFN-α in different and complementary ways. Both E2 and NS5A were capable of rescuing the replication of HCV replicon RNA during IFN-α treatment. Complete rescue was not observed as the amount of HCV RNA declined after 24 hours of IFN-α treatment (FIG. 4C).

PKR is not Induced but is Activated by dsRNA Present in Replicon-Containing Cells

PKR was fully inducible by IFN-α treatment in replicon-expressing cells (FIG. 4D) but PKR was not endogenously induced in the absence of IFN-α as indicated by the low level of PKR expression seen in untreated cells and the induction of PKR expression after IFN treatment. Endogenous IFN-α or IFN-β were, therefore, not being secreted by untreated Huh.BB7 cells. PKR activation was monitored by observing phosphorylation levels of the PKR substrate eIF-2α, (FIG. 4E) compared to the total amount of eIF-2α (FIG. 3E). In Huh7 cells eIF-2α was phosphorylated with the addition of IFN-α and dsRNA, but very little eIF-2α was phosphorylated in the absence of treatment (FIG. 4E, lanes 1, 2). This is consistent with the dsRNA-dependent activation of PKR. In Huh.BB7 cells, however, a high level of eIF-2α phosphorylation was observed in the absence of IFN-α and no additional phosphorylation was observed with the addition of dsRNA or IFN-α (FIG. 4E, lanes 3, 4). This indicates that although PKR was at a low basal-level of expression in untreated Huh.BB7 cells (FIG. 4D), it was already activated in replicon-containing cells (FIG. 4E, lanes 3, 4). Because no additional eIF-2α phosphorylation was observed after IFN-α and dsRNA treatment (FIG. 4E, compare, lanes 3 to 4), eIF-2α phosphorylation may have been already saturated. Alternatively, the assay itself may be saturated, allowing for no additional detection of eIF2α phosphorylation. To test this possibility and to evaluate the importance of PKR activation, we transfected the cells with the known PKR inhibitors HCV E2 (Taylor, D R et al. 1999 Science 285:107-110) or NS5A (Gale, M J et al. 1997 Virology 230:217-227) or both E2 and NS5A. All samples containing transfected E2 or NS5A demonstrated a decrease in the level of phosphorylated eIF-2α (FIG. 4E, lanes 5-10 compared to lanes 3 and 4), confirming that these two HCV genes inhibit PKR. Still, there was no additional phosphorylation of eIF-2α in the presence of IFN-α (FIG. 4E), even though translation decreased with IFN-α (FIG. 4A), and PKR protein expression increased in these cells (FIG. 4D), confirming that in these samples the assay was not saturated. These results also indicate that IFN-α treatment of replicon-containing cells does not lead to additional activation of PKR. This supports evidence demonstrated here and by others (Koev, G et al. 2002 Virology 297:195-202) indicating that not only PKR, but other pathways modulate IFN-α sensitivity of the replicon. It is consistent with the results showing that the PKR inhibitors can stimulate the replicon, but cannot overcome all the negative effects of IFN-α treatment (FIG. 4B, C, and E).

Because PKR was not fully induced (FIG. 4D), yet already activated in the cells expressing the replicon (FIG. 4E), dsRNA was most likely present, presumably from RNA replication of both positive- and negative-strand RNA. Taken together, these data are consistent with a stimulation of replicon expression by E2 and NS5A due to inhibition of PKR, which is activated by the replicon. Because IFN-α treatment was not required for activation of PKR (or its downstream effects) in replicon-containing cells (FIG. 4E) and yet, IFN-α is a potent antagonist of the replicon, a second IFN-α-induced antiviral pathway may be involved. This is also consistent with the results obtained in FIG. 3, where HCV IRES translation was insensitive to IFN-α. As shown previously by Koev et al. (Koev, G et al. 2002 Virology 297:195-202), PKR activation should result in the inhibition of both IRES-mediated and cap-mediated translation. Therefore, inhibition of the replicon by IFN-α may proceed through a secondary mechanism to PKR activation. These data indicate that the low level of PKR expression was sufficiently activated to enable phosphorylation of maximum levels of eIF-2α.

Activation of PKR probably occurred as a result of dsRNA present in replicon-containing cells but not in Huh7 cells alone. This is consistent with other findings showing that IFN-α regulatory factor 3 (IRF-3), which is important for initial induction of IFN-β in response to dsRNA or virus infection of cells, was inhibited in the presence of the replicon (Foy, E et al. 2003 Science 300:1145-1148). Usually, the activation of IRF-3 leads to the induction of IFN-β, and subsequently IFN-α/β-induced genes, such as PKR. We found that while dsRNA was present in replicon-containing cells, PKR expression was not induced, indicating that the dsRNA-induction of IFN-β was blocked, consistent with an inhibition of IRF-3.

VA RNA_{I} Confers IFN-α Resistance to the Replicon

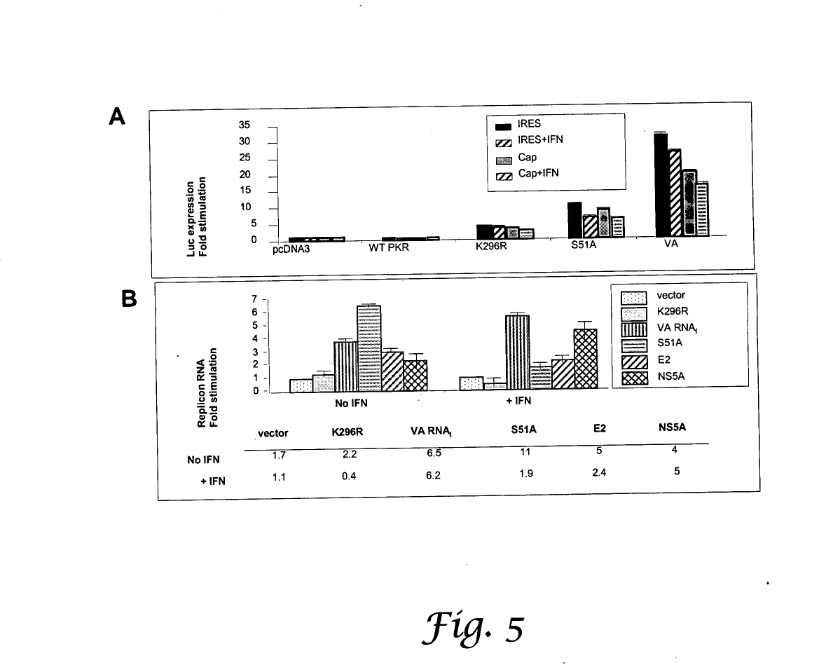

To examine the importance of eIF-2α phosphorylation in cellular and viral translation, we tested IFN-α sensitivity of a dual-Luc reporter. We used wild-type PKR1 (WT) and well-characterized PKR inhibitors to measure stimulation of translation, which was measured as an output of Luc expression. WT PKR, a catalytically inactive dominant-negative mutant PKR PKR K296R), an eIF-2α phosphorylation site mutant (eIF-2α S51A) that is non-phosphorylatable and VA RNA_{I} were cotransfected with the HCV IRES Luc reporter into Huh.BB7 cells. Over-expression of PKR (WT) inhibited expression of the Luc reporter, as expected (FIG. 5A), consistent with the behavior of transfected, active PKR (Taylor, D R et al. 2001 J Virol 75:1265-1273). Both PKR K296R and eIF-2α S51A stimulated Luc expression, even in the presence of IFN (FIG. 5A), indicating that eIF-2α phosphorylation was involved in the inhibition of both IRES-dependent and cap-dependent translation by IFN-α. Cotransfection with a DNA plasmid encoding VA RNA_{I} resulted in 25-fold stimulation of IRES-directed luc expression in the absence of IFN and nearly 20-fold stimulation in the presence of IFN (FIG. 5A). Cap-dependent translation was also stimulated by VA and to a higher degree than demonstrated by any of the other PKR inhibitors. While VA RNA is a potent inhibitor of PKR, this finding indicates that VA RNA_{I} can stimulate translation in replicon-containing cells by conferring IFN resistance through a pathway in addition to PKR. To examine the effects of these inhibitors on replicon RNA expression, we measured HCV replicon RNA in cells that were treated with IFN-α and transfected with the PKR inhibitors. Replicon expression was stimulated by all of the inhibitors, but most strongly in the presence of VA RNA_{I} and eIF-2α S51A (FIG. 5B). When these cells were treated with IFN-α, all inhibitors stimulated replicon expression over vector alone or the catalytically inactive K296R mutant. VA and NS5A, however, showed very efficient rescue of replicon RNA expression (FIG. 5B). Because the PKR inhibitors eIF-2α S5 1A and PKR K296R stimulated the replicon weakly (FIG. 5B), it is evident that PKR is involved in the limitation of the replicon in these cells. However, the robust stimulation of translation (FIG. 5A) and replicon RNA by VA RNA_{I} (FIG. 5B) indicates that a second potent antiviral pathway may be involved in the IFN-α -induced inhibition of the HCV replicon. Because VA RNA_{I} binds and inhibits ADAR1 in vitro (Lei, M et al. 1998 Virology 245:188-196), we looked for evidence of ADAR1 activity in replicon-containing cells.

IFN-α Results in A to I Mutations in Replicon RNA

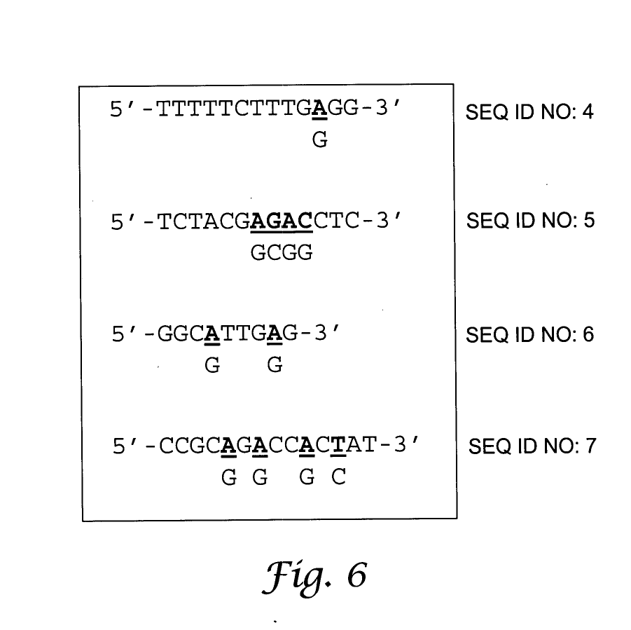

To examine evidence for editing in IFN-α-treated replicon cells, we sequenced RT-PCR products from cytoplasmic fractions of IFN-α-treated and untreated Huh.BB7 cells. If Adenosine-to-Inosine editing events occurred, then adenosine residues will read as guanosine residues. None of the clones from untreated cells, sequenced from 3 distinct regions of the replicon, diverged from the wild-type replicon sequence. However, one clone obtained from the IFN-α-treated cells contained mutations in adenosine residues resulting in guanosine (FIG. 6), indicating that the replicon RNA was directly edited. Because we had difficulty in obtaining PCR products that contained mutations, this indicates that once sequences are edited, they may not be replicated or they may be degraded.

VA RNA_{I} Impairs A-to-I Editing in Replicon Cells

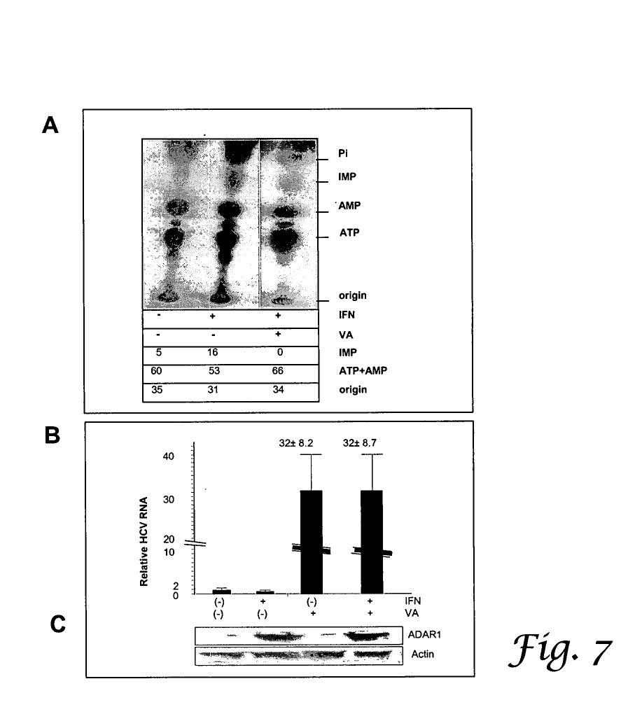

In addition to its inhibition of PKR, VA RNA_{I} is also known to inhibit ADAR1 (Lei, M et al. 1998 Virology 245:188-196). ADAR1 acts specifically on dsRNA, is IFN-α-inducible and causes the conversion of adenosine in dsRNA to inosine by deamination. To test the hypothesis that RNA editing occurs in replicon-containing cells, we examined the effects of IFN-α treatment on conversion of radiolabeled AMP to IMP. We isolated RNA that was metabolically labeled with α-³²P [ATP] from the cytoplasmic fraction of IFN-α-treated Huh.BB7 cells. Because VA RNA_{I} has been shown to specifically inhibit ADAR1 (Lei, M et al. 1998 Virology 245:188-196) and can also rescue IFN-α-inhibited replicon expression very efficiently (FIG. 5B), we wanted to know if VA was abrogating IFN-α sensitivity by inhibiting ADAR in replicon-containing cells. We measured radiolabeled IMP production in cytoplasmic RNA from Huh.BB7 cells transfected with VA RNA_{I} by PhosphorImager quantitation (FIG. 7A). Radiolabeled IMP was efficiently produced (16% of total adenosine products) in IFN-treated replicon cells. When we transfected the plasmid encoding VA RNA_{I}, the generation of radiolabeled IMP was inhibited to less than 1%, consistent with a role for VA in the inhibition of A-to-I editing by ADAR1 in IFN-treated replicon-containing cells. Previously, ADAR has been shown to convert approximately 40% of adenosines to inosine in dsRNA (Scadden, A D J and Smith, C W J 2001 EMBO J20:4243-4252).

In V RNA-transfected cells, the replicon was stimulated in the presence and absence of IFN-α (FIG. 7B) and yet ADAR1 was upregulated only when the cells were treated with IFN-α (FIG. 7C). This is consistent with previous observations whereby the replicon and Luc expression were stimulated by VA RNA in the absence of IFN-α in replicon cells (FIG. 5) and indicates that ADAR1 may already be active (as with PKR) due to dsRNA present in replicon-containing cells.

siRNA-Knockdown of ADAR1 Stimulates replicon expression

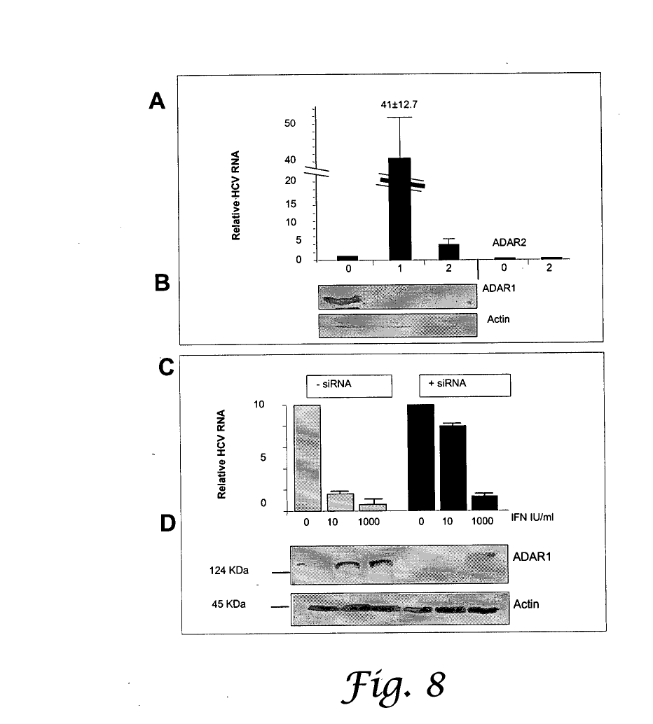

To confirm that the RNA editing occurring in replicon-containing cells was attributable to ADAR1, we used an RNA-interference assay utilizing small-inhibitory RNA (siRNA)-specifically directed to knockdown the expression of ADAR1 (Jayan, G C and Casey, J L 2002 J Virol 76:12399-12404). We used a siRNA that has been shown upon transfection to markedly decrease ADAR1 expression specifically (Jayan, G C and Casey, J L 2002 J Virol 76:12399-12404). Cells were transfected once and after 24 hrs, some were transfected for a second time. HCV replicon RNA increased with transfection of ADAR1 siRNA (FIG. 8A). At day seven post-transfection the HCV replicon RNA increased by 41-fold for one transfection and fivefold for two transfections of ADAR1 siRNA (FIG. 8A). Two transfections of ADAR1 siRNA were slightly toxic to the cells, which may attribute to the loss of activity of the siRNA. The results observed directly correlate with the expression level of the pl50, cytoplasmic, IFN-α -induced form of ADAR1 in cells (FIG. 8B). siRNA directed to ADAR2 knockdown (FIG. 8A) showed no stimulation of HCV replicon RNA, indicating that ADAR1 and not ADAR2 is responsible for instability of HCV replicon RNA.

The stimulation of the replicon by ADAR1 knockdown was seen in the absence of IFN-α (FIG. 8A, B). We, therefore, tested the effects of siRNA directed to ADAR1 in the presence of IFN. In the absence of siRNA transfection, IFN-α treatment resulted in a decrease in replicon RNA (FIG. 8C) corresponding with an increase in ADAR1 expression (FIG. 8D). Transfection with siRNA directed to ADAR1 resulted in loss of replicon RNA during IFN-α treatment as well (FIG. 8C). However, when compared to no-siRNA controls, the samples with siRNA yielded more replicon RNA (FIG. 8C), supporting the conclusion that IFN-α sensitivity of the replicon is mediated through ADAR1. The ADAR1 immunoblot (FIG. 8D) demonstrates that in the presence of IFN-α, siRNA does not completely knockdown ADAR1 expression, consistent with the observed replicon sensitivity in these samples.

Discussion

Our findings point to a new IFN-α -induced antiviral pathway important to the modulation of HCV replicon replication in cell culture. This pathway involves dsRNA-specific editing of adenosine residues by ADAR1 in HCV replicon-containing cells, which then leads to the loss of HCV replicon RNA. Viral RNA clearance may be attributable to one or several factors: first, editing and degradation of the viral RNA, possibly by an inosine-specific RNase (Scadden, A D J and Smith, C W J 1997 EMBO J 16:2140-2149; Scadden, A D J and Smith, C W J 2001 EMBO J 20:4243-4252), second, inefficient replication and genome instability may result from mutation of important viral sequences or third, a cellular message that is required for viral replication may be edited and inactivated. Taken together, our results explain why IFN-α action is specific for HCV-replicon RNA and not cellular RNA and how VA RNA_{I} can rescue replicon expression so effectively. These results confirm the findings that IFN-α action on the replicon is a result of both PKR and ADAR1 activation. Finally, the discovery that RNA editing negatively affects HCV RNA replication serves as the basis of a multitude of therapies that target this new IFN-α-induced antiviral pathway important for the clearance of HCV and other related viruses.

### EXAMPLE 1

### Expression Vectors and HCV Replicon

The BB7 replicon was a gift of C. M. Rice and K. Blight and was previously described (Blight, K J et al. 2000 Science 290:1972-1974). Briefly, the replicon expresses HCV NS3 through NS5B nonstructural genes under the encephalomyocarditis virus (EMCV) internal ribosome entry site (IRES) and neomycin resistance under the HCV IRES. The HCV 5′ and 3′ nontranslated regions are also present. Bicistronic reporter plasmids were constructed by substitution of the SV40 promoter in pRL-SV40 (Promega) with the HSV-1 alpha 27 promoter (gift of N. Martin). The mRNA expresses Renilla luciferase (Luc) through a cap-dependent translation mechanism and firefly luciferase under the EMCV IRES. PCR of the BB7 plasmid (using primers that contained a SalI restriction site at the 5′ end and a SacI restriction site at the 3′ end, covering nucleotides 1-386 in pHCVrep1bBB7) (Puig, M. et al. 2004 Vaccine 22:991-1000) was used to generate an HCV IRES cassette. The EMCV IRES was removed by digestion with SalI and SacI and the HCV-IRES was inserted. pcDNA3 (Invitrogen) plasmids expressing wild-type PKR (WT), PKR K296R, E2 and NS5A were described previously (Taylor, D R et al. 1999 Science 285:107-110). eIF2α-pRc/CMV (eIF-2-α S51A) was a gift of O. Donze and was described previously (Donze, 0 et al. 1995 EMBO J 14:3828-3834). VA RNA_{I}-pUC119, expressed under the polymerase III intragenic promoter was previously described (Gunnery, S 1995 Mol Cell Bio 15:3597-3607).

Cell Culture

Huh7 and Huh.BB7 cells were grown at 37° C. in Dulbecco's Modified Eagle's Medium (DMEM) with 10% fetal bovine serum, 2 mM L-glutamine, and 100 U/ml penicillin, and 100 μg/ml streptomycin. Huh7 cells were transfected with RNA transcribed from linearized pHCVrep1bBB7 plasmid. Stable transfectants were selected in Geneticin (G418, Gibco), and grown in G418 at 75 μg/mL, which was removed during experiments. Cellular proteins were metabolically labeled in cell culture by incubating for 1 hour at 37° C. in DMEM minus methionine and then supplementing for one hour with ³⁵S-methionine. RNA was metabolically labeled in cell culture by incubating the cells for 1 hour at 37° C. in DMEM minus phosphates and then incubating the cells for 16 hours in medium supplemented with α-³²P [ATP]. Cell growth was monitored by counting viable cell number with trypan blue staining or by Coulter Counter (Beckman Coulter, Inc.). Cells were treated with recombinant human IFN-α2A (100 IU/mL in DMEM) for 18 hours at 37° C., unless stated otherwise.

Analysis of Protein and RNA

Cells were washed with PBS and the attached monolayer was incubated with trypsin EDTA for five minutes at 37° C. Cells were collected, concentrated in 0.1% NP-40 and 10% PBS, and lysed by three repeated freeze-thaw cycles. Nuclear and cellular membranes were removed by centrifugation and cytoplasmic extracts were quantitated for protein concentration with a colorimetric absorbance protein assay (Bio-Rad). Expression of HCV NS3 protein in lysates from Huh7 or Huh.BB7 cells was monitored by resolving 25 μg of protein extract in SDS-PAGE and immunoblotting with polyclonal antibody to NS3 (Chimp 1536 serum; Puig, M. et al. 2004 Vaccine 22:991-1000). Polyclonal anti-PKR antiserum was made by inoculating New Zealand White rabbits with a keyhole limpet hemocyanin-conjugated peptide to the spacer region between the two dsRNA-binding motifs of PKR (peptide P1; Taylor, D R et al. 2001 J Virol 75:1265-1273). E2 expression was monitored with mouse monoclonal antibody Al1 using cell extracts that were treated with endoglycosidase H. Actin expression was detected using polyclonal goat anti-actin antibodies (Santa Cruz Biotechnology). Monoclonal antibodies (Cell Signaling Technology) were used to detect phospho-eIF-2α and total eIF-2α proteins from 20 pg of protein from the cytoplasmic fraction (as determined by Bio-Rad protein assay) of cells that were harvested as described above with the addition of phosphatase inhibitors (90 mM sodium fluoride, 17.5 mM sodium molybdate, 17.5 mM β-glycerophosphate).

Cellular RNA was isolated using the Trizol method (Gibco) and quantitated by ultraviolet absorbance at 260 nm. HCV replicon RNA from 0.5×10⁶ cells was monitored by real-time RT-PCR (Taqman, Applied Biosystems) using primers located on the HCV 5′ end and quantified based on HCV RNA standards and relative amounts of cellular GAPDH messenger RNA as described previously (Wong, S K et al. 2003 RNA 9:586-598).

RNase protection analysis was performed using the Hybspeed RPA kit (Ambion) to examine the transfection efficiency of luciferase reporters. Cellular RNA was isolated and incubated with an antisense RNA probe to 125 nucleotides of Renilla luciferase, synthesized with T7 RNA polymerase and [α-³²P]dCTP. The assay was performed as per the manufacturer's suggestions. RNA was quantitated using known RNA concentrations of the sense strand of the probe, synthesized with SP6 RNA polymerase.

Luc Assays

Luciferase assays were performed with a Dual Luciferase Reporter System (Promega; as per the manufacturer's directions) and Luc activity was measured with luminometer (Turner Designs). Lysates were prepared from Huh7 or Huh.BB7 cells 48 hours after transfection with a bicistronic reporter plasmid (2 μg DNA/transfection) or bicistronic Luc reporter RNA (synthesized in vitro with T7 RNA polymerase after linearization of the plasmid). Luc assays shown are representative of 4 or more experiments with transfections performed in duplicate and samples tested in the luc assay in triplicate. Similar results were obtained in all experiments.

Thin Layer Chromatography

Radioactive monophosphates were resolved by thin layer chromatography (TLC) on polyethylenimine cellulose developed in ammonium acetate buffer as described previously (Scadden, A D J and Smith, C W J 2001 EMBO J20:4243-4252). Briefly, RNA was digested with nuclease P1 (Roche) after Trizol isolation from Huh.BB7 cells that were grown in the presence of [α-³²P]ATP. Nonradioactive AMP and IMP were used as migration standards and visualized with UV light. Radioactivity was visualized by autoradiography and quantitated by Phosphorlmage analysis.

siRNA

Huh.BB7 cells were plated at 1×10⁶ cells/ml. Huh.BB7 cells were transfected with ADAR1 or ADAR2 small interfering RNA (siRNA) (final concentration of 100 nM) (a gift of John Casey) as described previously (Vyas, J. et al. 2003 RNA 9:858-870) with DMRIE-C transfection reagent (Invitrogen). Cells were transfected with ADAR siRNA once (1×) or transfected twice (24 hr later) and harvested seven days after initial transfection.

Isolation and Sequencing of Replicon RNA

Huh.BB7 cells were treated with IFN-α (100 IU/ml, 18 hr) and cytoplasmic RNA was extracted by disrupting cells in 0.1% NP-40, O.1× PBS (10%) as described above. RT was performed using First strand cDNA kit (Amersham) with a primer in the antisense direction of the NS5 region (5′-CAACCGTCCTCTTCCTCCG-3′, SEQ ID NO: 1) and PCR was performed using Expand high fidelity PCR system (Roche) with primers directed towards the HCV IRES region (5′-GCATGCGTCGACGCCAGCCCCGGATTGGGG-3′, SEQ ID NO: 2 and 5′-AGGTCGAGCTCGGCGCGCCCTTTGGTTTTTC-3′, SEQ ID NO: 3). PCR products were inserted into pCR II-Topo with a Topo TA cloning kit (Invitrogen, white clones were selected, and DNA was purified and sequenced with the forward primer included in the kit.

### EXAMPLE 2

### Inhibition of ADAR1 in IFN-α-Deficient Cells and Permissive Culture of HCV In Vitro

Cell Tropism

Methods: Cells were cultured to provide an overnight confluency of 35%. The cells were then transfected with plasmid vector pVA/s6 (which is essentially puc 119, containing the Adenovirus type 2 VA RNA_{I} sequence, described in Gunnery and Mathews 1995 Molecular and Cellular Biology 15:3597-3607 using Dmrie-C (Invitrogen). These cultures were maintained overnight at 37° C. in DMEM containing FBS (Biofluids). The following morning the cultures were infected with 5×10⁴ copies of RNA in serum from a Hepatitis C virus chronically-infected Chimpanzee (Ch 1536). When the Vero cultures reached confluency, they were harvested. The cells sheets were washed in PBS and trypsinized (Biofluids). The cell lysate was then washed after spinning with PBS. NP-40 (Sigma) in PBS was added to the washed pellet and three freeze thaw cycles lysed the cells. Centrifugation was implemented to remove cellular debris. Nested PCR was done on the supernatant for evidence of HCV.

Results: Only Vero cells transfected with VA were positive for HCV RNA in the first round of PCR (Table 1A). Positive results in the first round of PCR indicate that the amount of viral RNA exceeds 10⁴ copies of RNA/ml, which we have detected by Taqman quantitation. When the PCR products were amplified by nested PCR, the HHT4 and lymphocytes transfected with VA were positive as well. This indicates that primary liver and lymphocytes may be permissive to HCV infection, but that robust growth of the virus requires VA RNA and preferably in Vero cells.

TABLE 1A
Vero cells can be made permissive for HCV with VA

| Cells | Transfection | PCR | Nested PCR |
|---|---|---|---|
| PLC (HHT4) | no | (−) | (+) |
| Vero | no | (−) | (−) |
| Vero | VA | (+) | (+) |
| Human lymphocytes | no | (−) | (−) |
| Human lymphocytes | VA | (−) | (+) |
| B958 (marmoset lymphocytes) | no | (−) | (−) |
| B958 (marmoset lymphocytes) | no | (−) | (−) |

Persistent Infection

Methods: The following changes were made to the methods described for Table 1A: After trypsinization the infected Veros were washed with PBS, resuspended in PBS, and a small volume of cells (split ⅙) were removed for transfection with VA and then were plated. This process was repeated with the same culture for 20 weeks. The remaining cells were processed for HCV/PCR testing.

Results: Cells that were transfected with VA were positive at low titer for 5 weeks (Table 1B). At week 6 the titer increased (as demonstrated by positive results in the first PCR). Titer remained high and dropped off at week 13. The cells were very weakly positive until week 18. This indicates that HCV can be maintained in cell culture and can possibly be maintained long enough to obtain tissue culture adaptive strains.

TABLE 1B
Vero cells with VA can be persistently infected

| week | PCR/Nest |
|---|---|
| 1 | −/+ |
| 2 | −/+ |
| 3 | −/+ |
| 4 | −/− |
| 5 | −/− |
| 6 | +/+ |
| 7 | +/+ |
| 8 | ±/+ |
| 9 | ±/+ |
| 10 | ±/+ |
| 11 | ±/+ |
| 12 | ±/+ |
| 13 | −/+ |
| 14 | −/− |
| 15 | −/− |
| 16 | −/− |
| 17 | −/− |
| 18 | −/+ |
| 19 | −/− |
| 20 | −/− |

Vero Cells can be Infected with HCV if Transfected with VA

Methods: Kinetics of infection was determined by infecting cells with and without VA RNA.

Results: Those cells that contained VA demonstrated the presence of HCV RNA in the first PCR reaction at day 1, which persisted until day 10 (Table 2). Titer started dropping off at day 13.

TABLE 2
VERO cells with VA can be infected (kinetics)

| Day | VERO | VERO & HCV | VERO & VA & HCV |
|---|---|---|---|
| 1 | −/+ | −/+ | +/+ |
| 3 | −/− | −/+ | +/+ |
| 6 | −/− | −/+ | +/+ |
| 7 | −/− | −/− | +/+ |
| 10 | −/+ | −/− | +/+ |
| 13 | −/− | −/− | −/+ |

Sequencing of the Replicon and A to G Mutations

Methods: Replicon-containing Huh7 cells were treated with interferon (100 μ/ml) for 18 hours and RNA was harvested from the cytoplasmic fraction. RT-PCR was performed with specific HCV primers and then nested PCR was performed. PCR products were cloned into TA vectors and sequenced.

Results: Most clones were found to have wild type HCV sequences. We detected A to G mutations in one clone. This is shown in Table 3.

Table 3. Sequencing of BB7 Replicon after IFN Treatment Yields A to G Mutations

| | |
|---|---|
| TTTTTCTTTGAGG | (SEQ ID NO: 4) |
| G | |
| TCTACGAGACCTC (SEQ ID NO: 5) | |
| GCGG | |
| GGCATTGAG (SEQ ID NO: 6) | |
| G G | |
| CCGCAGACCACTAT (SEQ ID NO: 7) | |
| G G G C | |

Conclusions

Our data show that Vero cells are permissive for HCV infection. Vero cells are known to be deficient for interferon production, due to the lack of interferon genes. We also show that VA stimulates the growth of HCV in culture. VA RNA is known to circumvent the interferon response through PKR and ADAR1. Taken together, our results show that by using cells that are defective in interferon production, and further inhibiting the interferon-induced antiviral pathways, we can achieve growth of HCV in culture. We believe that the interferon-induced pathways are the limiting factor in HCV growth in cell culture.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

What is claimed is:

1. A composition of matter comprising a cell culture infected with an animal virus, wherein said cell culture is an interferon-deficient cell culture that is deficient in adenosine deaminase that acts on dsRNA (ADAR1) activity.

2. The composition of claim 1, wherein said ADAR1 -deficient cells are obtained by transfection of a parent cell culture with a small-inhibitory RNA specifically directed to knockdown the expression of ADAR1.

3. The composition of claim 1, wherein said ADAR1-deficient cells are obtained by transfection of a parent cell culture with an ADAR1 antisense polynucleotide.

4. The composition of claim 1, wherein said ADAR1 -deficient cells are obtained by transfection of a parent cell culture with an ADAR1 dominant negative mutant gene.

5. The composition of claim 1, wherein said ADAR1-deficient cells are obtained by treatment of a parent cell culture with a small organic or inorganic molecule that inhibits ADAR1.

6. The composition of claim 1, wherein said ADAR1-deficient cells are obtained by transfection of a parent cell culture with adenovirus associated RNA (VA RNA).

7. The composition of claim 1, wherein said ADAR1-deficient cell culture is derived from a cell line selected from the group consisting of NALL-1, K562, R24;1 1, Reh, SUP-T3, K-T1 and Vero cells.

8. The composition of claim 7, wherein said cell line is Vero cells.

9. The composition of claim 1, wherein said animal virus is an RNA virus.

10. The composition of claim 9, wherein said RNA virus is a hepatitis C virus.

11. A method for production of a viral vaccine for an animal virus comprising:
(a) infecting a cell culture with a donor virus, wherein said cell culture is an interferon-deficient cell culture that is deficient in ADAR1 activity;
(b) culturing said infected cell culture under conditions sufficient to provide efficient virus growth; and
(c) harvesting the virus produced.

12. The method of claim 11, wherein said ADAR1-deficient cells are obtained by transfection of a parent cell culture with a small-inhibitory RNA specifically directed to knockdown the expression of ADAR1.

13. The method of claim 11, wherein said ADAR1-deficient cells are obtained by transfection of a parent cell culture with an ADAR1 antisense polynucleotide.

14. The method of claim 11, wherein said ADAR1-deficient cells are obtained by transfection of a parent cell culture with an ADAR1 dominant negative mutant gene.

15. The method of claim 11, wherein said ADAR1 -deficient cells are obtained by treatment of a parent cell culture with a small organic or inorganic molecule that inhibits ADAR1.

16. The method of claim 11, wherein said ADAR1-deficient cells are obtained by transfection of a parent cell culture with adenovirus associated RNA (VA RNA).

17. The method of claim 11, wherein said ADAR1-deficient cell culture is derived from a cell line selected from the group consisting of NALL-1, K562, R24; 11, Reh, SUP-T3, K-T1 and Vero cells.

18. The method of claim 17, wherein said cell line is Vero cells.

19. The method of claim 11, wherein said animal virus is an RNA virus.

20. The method of claim 19, wherein said RNA virus is a hepatitis C virus.

21. A method for determining the antiviral activity of a compound against an animal virus, said method comprising:
(a) infecting a cell culture with animal virus, wherein said cell culture is an interferon-deficient cell culture that is deficient in ADAR1 activity;
(b) treating said infected cell culture with said compound;
(c) culturing said infected cell culture under conditions sufficient to provide efficient virus growth; and
(d) determining the yield of virus produced.

22. A method for determining the antiviral activity of a compound against an animal virus, said method comprising:
(a) infecting a susceptible host cell culture with an animal virus;
(b) treating said infected culture with said compound;
(c) preparing a cell extract of said treated cell culture;
(d) exposing under infective conditions an indicator cell culture to said cell extract, wherein said indicator cell culture is an interferon-deficient cell culture that is deficient in ADAR1 activity;
(e) culturing said exposed cell culture under conditions sufficient to provide maximum virus growth; and
(f) determining the yield of virus produced.

23. A method for determining the antiviral activity of a compound against an animal virus, said method comprising:
(a) treating said animal virus with said compound;
(b) exposing under infective conditions an indicator cell culture with said treated animal virus, wherein said indicator cell culture is an interferon-deficient cell culture that is deficient in ADAR1 activity;
(c) culturing said exposed cell culture under conditions sufficient to provide maximum virus growth; and
(d) determining the yield of virus produced.

24. A method for detecting the presence of virus in a sample comprising:
(a) exposing under infective conditions an indicator cell culture to said sample suspected of containing a virus, wherein said indicator cell culture is an interferon-deficient cell culture that is deficient in ADAR1 activity;
(b) culturing said exposed cell culture under conditions sufficient to provide for maximum virus growth; and
(c) analyzing said cell culture for the presence of said virus.

25. A combination for treating patients having infection by hepatitis C virus or another related virus for a time sufficient to eradicate detectable virus for at least 24 weeks after the end of a period of administration comprising a therapeutically effective amount of an interferon-α and a therapeutically effective amount of an ADAR1 agonist.

26. A method of treating patients having infection with hepatitis C or another related virus comprising administering a therapeutically effective amount of interferon-α and a therapeutically effective amount of an ADAR1 agonist for a time sufficient to eradicate detectable virus for at least 24 weeks after the end of said period of administrating.


SEQUENCE LISTING (7 sequences)

SEQ ID NO: 1 - DNA, 19 bp, Artificial Sequence
  Synthetic primer
caaccgtcctcttcctccg

SEQ ID NO: 2 - DNA, 30 bp, Artificial Sequence
  Synthetic primer
gcatgcgtcgacgccagccccggattgggg

SEQ ID NO: 3 - DNA, 31 bp, Artificial Sequence
  Synthetic primer
aggtcgagctcggcgcgccctttggtttttc

SEQ ID NO: 4 - DNA, 13 bp, Artificial Sequence
  artificial replicon
tttttctttgagg

SEQ ID NO: 5 - DNA, 13 bp, Artificial Sequence
  artificial replicon
tctacgagacctc

SEQ ID NO: 6 - DNA, 9 bp, Artificial Sequence
  artificial replicon
ggcattgag

SEQ ID NO: 7 - DNA, 14 bp, Artificial Sequence
  artificial replicon
ccgcagaccactat